(19) United States
(12) Patent Application Publication
(10) Pub. No.: US 2016/0067298 A1
(43) Pub. Date: Mar. 10, 2016

(54) MUSCADINE GRAPE SKIN EXTRACT AS TREATMENT FOR BONE METASTATIC CANCER

(71) Applicant: Clark Atlanta University, Atlanta, GA (US)

(72) Inventor: Valerie Odero-Marah, Tucker, GA (US)

(21) Appl. No.: 14/850,718

(22) Filed: Sep. 10, 2015

Related U.S. Application Data
(60) Provisional application No. 62/048,328, filed on Sep. 10, 2014.

(51) Int. Cl. A61K 36/87

(52) U.S. Cl. CPC A61K 36/87

(57) ABSTRACT

Muscadine Grape Skin Extract (MSKE) derived from muscadine grape (Vitis rotundifolia) decreases Snail expression and CatL expression and activity and pSTAT-3. MSKE inhibits migration and invasion and osteoclastogenesis of cancer cells.

### CROSS-REFERENCE TO RELATED APPLICATIONS

This application is based upon and claims the benefit of priority from U.S. provisional application No. 62/048,328 filed Sep. 10, 2014, the entire contents of which are hereby incorporated herein by reference.

### STATEMENT REGARDING FEDERALLY SPONSORED RESEARCH OR DEVELOPMENT

This invention was made with government support under grants G12RR003062-22 and P20MD002285-01 awarded by the NIH. The government has certain rights in the invention.

### BACKGROUND

Muscadine Grape Skin Extract (MSKE) is derived from the muscadine grape (Vitis rotundifolia). Based on the skin color, muscadine varieties are referred to respectively as bronze and purple compared to white and red for all other grapes. Muscadine grapes are native to the Southeastern United States and can be found growing wild from Delaware to the Gulf of Mexico and westward from Missouri to Texas. Although a few studies have reported high polyphenols content of muscadine grapes, little research has been conducted to evaluate the bioactivities of the phenolic compounds in any muscadine grape. Muscadine grapes possess one of the highest antioxidant levels among fruits. This grape has been shown to decrease inflammation (decreasing IL-6) and promote apoptosis in prostate cancer cells by decreasing Akt and MAPK signaling pathways; it has also been shown to revert the epithelial-mesenchymal transition by increasing the expression epithelial markers such as E-cadherin and decreasing the expression of mesenchymal markers such as vimentin and Snail. MSKE is currently in Phase II Clinical Trials at John Hopkins University to test if it can lower prostate specific antigen (PSA) levels, a protein used as a biomarker for prostate cancer. However, the effects of this natural product have never before been tested on the formation of mature osteoclasts that play a vital role in metastasis.

Breast and prostate cancer are a leading cause of cancer death among women and men. The skeleton is a preferred site for breast and prostate cancer metastasis. More than 80% of all men who die of prostate cancer have metastatic disease within the bone. Osteoblastic lesions, characteristic of prostate cancer, are caused by an excess of osteoblast activity relative to resorption by osteoclasts, leading to abnormal bone formation. In breast cancer, osteolytic lesions are found in 80% of patients with stage IV metastatic disease. The lesions are characterized by increased osteoclast activity and net bone destruction. The primary cause of prostate and breast cancer death is metastasis, which is regulated by several factors and signaling pathways such as epithelial mesenchymal transition (EMT), a dynamic process that promotes cell motility with decreased adhesive ability. Snail, a zinc-finger transcription factor, has been found to regulate EMT in part by increasing extracellular matrix (ECM) degradation via up-regulation of matrix metalloproteinases (MMPs). STAT3 signaling has been shown to increase Snail expression through Liv-1 zinc transporter.

Previous reports have shown that ARCaP and LNCaP prostate cancer cells stably transfected with Snail displayed decreased adhesion and increased cell migration. It has also been shown that receptor activator of NFkB ligand (RANKL), a member of the TNF family that is normally expressed on the cell surface of stromal cells and osteoblasts and mediates osteoclast differentiation and osteolysis or bone resorption, can be up-regulated by Snail overexpression in ARCaP and LNCaP prostate cancer cells, which was associated with increased osteoclastogenesis in vitro and in vivo. Acidosis of the bone microenvironment results in increased osteoclast resorption pit formation with osteoclasts being maximally stimulated at pH levels less than 6.9. Acidosis alters cellular dynamics at the interface between the tumor and normal tissue, promoting apoptosis in adjacent normal cells and facilitating extracellular matrix degradation through the release of proteolytic enzymes such as Cathepsins B, D, and L which degrade the extracellular matrix and facilitate metastasis.

Cathepsins are cysteine proteases belonging to the papain family of peptidases. Currently 11 cysteine cathepsins have been identified including cathepsins K, L, S and V, which have been implicated in a number of pathological diseases including atherosclerosis, abdominal aortic aneurysms, osteoporosis and arthritis, and colon and breast carcinomas. Cysteine cathepsins are primarily intracellular proteases that function in terminal protein degradation in lysosomes and protein processing in other intracellular organelles. Cathepsins have been shown to have specific roles in bone remodeling and cancer progression by increasing invasion. Mature osteoclasts secrete proteinases such as Cathepsin K and MMP-9, which are needed to degrade the organic matrix of bone in the microenvironment of low pH. Cathepsins are proteases that play a role in ECM degradation, but no direct link has ever been shown between Snail and cathepsins. Cathepsin L (CatL) is a cysteine cathepsin that is overexpressed in a variety of cancers such as lung, colon, breast and prostate cancer, and is also involved in the repression of E-cadherin, a hallmark of epithelial mesenchymal transition (EMT). CatL is either secreted or associated with the plasma membrane and degrades the extracellular matrix during tumor progression. Procathepsin L and processed mature CatL can degrade laminin and fibronectin extracellular matrices, while Cat L can also degrade collagen in vitro. Treatment options for metastatic cancers are associated with adverse side effects and a risk for tumor recurrence. Although inhibitors of CatK have been used in clinical trials for osteoporosis and breast cancer, there are no CatL inhibitors in clinical trials and even with CatK inhibitors, there has been concern about off-target effects involving the danger in targeting non-osteoclast related functions of CatK.

Studies have suggested that fruit and vegetables can have chemopreventive and therapeutic effects on tumor cells. Muscadine grape skin extract (MSKE) with anthocyanin as the main bioactive component has shown its ability to inhibit prostate cancer cell growth and promote apoptosis in vitro without toxicity to normal prostate epithelial cells.

We have shown that CatL expression increases with tumor grade in prostate and breast patient tissue. Additionally, Snail overexpression increases CatL activity and treatment with MSKE leads to a decrease in Snail and CatL activity.

### SUMMARY OF THE INVENTION

Snail transcription factor expression is increased in prostate cancer and associated with increased invasion, migration, and bone turnover. Cathepsin L (CatL) is a cysteine cathepsin protease that is overexpressed in cancer and involved in bone turnover. We observed an increase of CatL expression in prostate and breast tumor tissue compared to normal tissue. We also tested the expression and activity of CatL in breast (MCF-7) and prostate (LNCaP, ARCaP-E) cells overexpressing Snail or C4-2 (the aggressive subline of LNCaP) with stable Snail knockdown. Snail overexpression led to increased CatL and phosphorylated STAT-3 (pStat-3), compared to Neo vector controls, while the reverse was observed in cells with Snail knockdown.

Muscadine Grape Skin Extract (MSKE) derived from muscadine grape (Vitis rotundifolia) decreased Snail expression and abrogated Snail-mediated CatL activity and pSTAT-3. Functionally, cancer cells overexpressing Snail displayed increased migration and invasion and osteoclastogenesis, which were significantly inhibited by the addition of MSKE. Taken together, these findings suggest that bidirectional signaling between Snail and CatL activity occurs via Stat-3 signaling and can be antagonized by MSKE possibly acting in part through CatL inhibition. Therefore MSKE could potentially be a promising bioactive compound for metastatic cancer.

### BRIEF DESCRIPTION OF THE DRAWINGS

FIGS. 1A, 1B and 1C illustrate that Snail overexpression increases Cathepsin L expression/activity. FIG. 1A shows Western blot analysis and FIGS. 1B and 1C show zymography results.

FIGS. 2A, 2B and 2C are Western blot analysis (FIG. 2A) and zymography (FIG. 2B, FIG. 2C) showing that STAT3 knockdown decreases Snail and Cathepsin L expression/activity.

FIG. 3 illustrates the effects of MSKE and CatL inhibition on Snail-mediated CatL activity utilizing various cells that have been stably transfected to over-express Snail transcription factor or stably have a knockdown of Snail analysis of mature cathepsin L expression and activity. FIG. 3A is western blot analysis and FIG. 3B is zymography results.

FIG. 4 illustrates the effects of MSKE and Z-FY-CHO on cell migration and invasion.

FIG. 5 shows the effects of MSKE and Z-FY-CHO on osteoclastogenesis in prostate cancer cells.

FIG. 6 shows the effects of MSKE and Z-FY-CHO on osteoclastogenesis in breast cancer cells.

FIG. 7 shows cell migration in C4-2 parental prostate cancer cells displayed decreased cell migration upon treatment with MSKE.

### DETAILED DESCRIPTION OF THE INVENTION

Several types of cancers are deeply linked with the skeleton and cause an increase in osteoclast formation, which ultimately leads to bone metastasis. Bone metastasis makes bone more fragile and leads to pathologic fractures and spinal compression. This osteolysis is associated with severe bone pain, which may be intractable. Bone metastasis represents a common cause of morbidity in patients with many types of cancer, occurring in as many as 70% of patients with advanced breast or prostate cancer. The presence of an osteolytic component in prostate cancer skeletal metastases suggests that osteoclastogenesis may play a role in the establishment of these lesions. Recently, the discovery of the TNF family member, receptor activator of NF-KB (RANKL); its receptor, receptor activator of NF-KB (RANK); and its decoy receptor, osteoprotegerin (OPG) has established a common mechanism through which osteociastogenesis is regulated in normal bone. RANKL, a transmembrane molecule located on bone marrow stromal cells and osteoblasts, binds to RANK, which is located on the surface of osteoclast precursors. This ligand-receptor interaction activates NF-KB, which stimulates differentiation of osteoclast precursors to osteoclasts. OPG, also produced by osteoblasts/stromal cells, binds to RANKL, sequestering it from binding to RANK, which results in inhibition of osteoclastogenesis.

The requirement for RANKL to induce osteoclastogenesis suggests that it may mediate the osteolytic component of prostate cancer skeletal lesions. Previous work has shown that Snail transcription factor when overexpressed ARCaPE cells are able to increase the expression of RANKL and the formation of osteoclasts in vitro and in vivo. This Snail-induced RANKL provides a crucial link between EMT and possible bone turnover in prostate cancer. We have also recently identified that Snail can increase activity of cysteine protease Cathepsin L (CatL) that may be involved in bone resorption.

CatL is an endopeptidase that is able to perform limited proteolysis in the endosomes and lysosomes of specific cell types. There are also reports of Cad, working in the nucleus and cleaving CDP/CUX transcription factor. The aim of this report is to show that CatL is important in cancer progression and metastasis and can be regulated by Snail transcription factor, and that

Snail signaling and CatL activity can be antagonized with MSKE. CatL expression increases with prostate cancer progression. Although these cathepsin proteases are mostly secreted, the mechanism(s) by which they are upregulated in prostate or cancer has not been elucidated. We have previously shown by immunohistochemistry (IHC) that Snail expression is higher in aggressive and bone metastatic prostate cancer patient tissue and that Snail can promote osteoclastogenesis in vitro and in vivo. It has also been indicated that Snail-positive breast cancer tends to home to the bone in breast cancer patients. In our present tissue microarray samples we show that CatL is highly expressed with advanced stages of prostate cancer and that the expression of CatL shifts from predominantly cytoplasmic in lower grade to nuclear in higher grade tumor tissue. In normal vs. tumor matched lysates it is also shown that the mature Cat L is expressed at a higher amount in tumor lysates as compared to normal tissue. Nuclear localization of CatL has previously been documented using in vitro cultures and has been found to have distinct DNA binding and transcriptional regulatory activities. In these studies, a truncated form of CatL cleaves the CUX1 transcription factor and as a result accelerates progression into the S phase of the cell cycle. CatL is also located in the nucleus of breast cancer cells and patients with triple negative breast cancer have a higher levels of nuclear CatL. We show also that with increasing progression of prostate cancer that CatL is expressed in the nucleus, which may infer that Cat L activity in the nucleus is associated with a poor prognosis in prostate cancer. We also show that Snail increases CatL expression and activity. This is the first report showing that Snail can regulate CatL expression/activity.

Constitutive activation of STAT-3 has been observed in many human tumors including prostate. When we knocked down STAT-3 in cells overexpressing Snail there was a decrease in Snail and mature CatL expression, and CatL activity. This indicates that Snail activates CatL via the STAT-3 signaling pathway. We also present novel findings that MSKE inhibits the activity of CatL by inhibiting Snail expression. MSKE has been shown previously to promote apoptosis of prostate cancer cells without affecting normal prostate epithelial cells. We have also shown that MSKE can antagonize Snail-mediated EMT. After treatment with MSKE for 72 hours we observed that MSKE decreased Snail expression as well as CatL and STAT-3 activity in cells over expressing Snail. MSKE may antagonize Snail-mediated signaling by inhibiting the JAK/STAT pathway. We found that MSKE could also abrogate the Snail-mediated functional increase in cell migration, invasion, and osteoclastogenesis in both prostate and breast cancer cells.

Therefore, we show here for the first time that Snail mediates migration, invasion, and osteoclastogenesis in part via CatL. Of note is that although both CatL inhibitor and MSKE both antagonized osteoclastogenesis, MSKE appeared to significantly decrease the number of cells as compared to CatL inhibitor. This is not surprising, as 20 MSKE apoptosis. Additionally, although MCF-7 Snail cells displayed a significantly higher number of mature osteoclasts as compared to MCF-7 Neo, there were hardly any cancer cells noted on the MCF-7 Snail plate following TRAP staining. MCF-7 Snail cells generally attach very loosely and we believe this is due to Snail decreasing cell adhesion, therefore, the cells tend to be easily washed off. CatL inhibition is already being discussed as a possible therapy for bone metastasis, but this is the first study suggesting that MSKE may also be a potential therapy for bone metastatic disease.

Overall, this study develops novel roles for bidirectional interactions between Snail transcription factor and CatL that involves STAT-3 signaling. Although the underlying mechanisms governing these effects are not yet fully understood, the available evidence collectively indicates that MSKE may be of therapeutic benefit in clinical settings, suggesting its potential use as an anticancer agent or an adjunct to current cancer therapies.

We obtained MSKE from Dr Tamaro Hudson, our collaborator from Howard University and used it to perform osteoclastogenesis assay on prostate and breast cancer cell lines, including one in which Snail, a transcription factor, is overexpressed. 3×10³ ARCaP-Neo/MCF-7 Neo or ARCaP-Snail/ MCF-7 Snail was co-cultured with 40×10⁴ spleen macrophages in 48-well plates plus 1 ng/ml M-CSF plus or minus 5 μg/ml MSKE, 20 μg/ml MSKE, and 5 μM Z-FY-CHO CatL inhibitor.

The cells were fed every three days by replacing half the media with fresh media plus or minus MSKE or Z-FY-CHO. Macrophages alone were utilized as a negative control. After 7-14 days the cells were fixed with 3% formaldehyde and processed for TRAP staining according to manufacturer instructions, to visualize the formation of mature osteoclasts.

The cell lines used included ARCaPE prostate cancer and MCF-7 breast cancer cells stably transfected with either an empty vector (Neo) as a control or Snail transcription factor cDNA. With these steps we were able to get quantitative data for the formation of osteoclast with treatment of these cell lines with MSKE. We found that the 5 μM Z-FY-CHO CatL inhibitor, 50 ng/ml OPG, 5 μg/ml MSKE, and 20 μg/ml MSKE significantly decreased the formation of osteoclast compared to the untreated control.

We also performed Western blot analysis of whole cell lysates obtained from untreated control and MSKE treatments. ARCaPE and MCF-7 cells over expressing Snail were treated with MSKE for 3 days, and probed for Snail, phosph-STAT-3 (p-STAT3), and Callusing Western blot analysis.

With these steps we were able to detect higher protein expression of Snail, CatL and p-STAT3 in cells overexpressing Snail as compared to Neo empty vector control cells. The treatment with MSKE led to a decrease in the expression of Snail, STAT-3, p-STAT-3, and CatL expression and activity. These results indicate that MSKE is capable of targeting important pathway signals that may be crucial in the formation of osteoclasts and ultimately bone metastasis.

We also performed cathepsin zymography to detect cathepsin activity in untreated control and MSKE treatments. ARCaPE and MCF-7 cells over expressing Snail were treated with MSKE for 3 days using the condition media from the cell lysates. With these steps we were able to detect higher CatL activity (enzymes hydrolyze the embedded substrate in situ, and proteolytic activity can be visualized as cleared bands) in cells overexpressing Snail as compared to Neo empty vector control cells. We observed that the 5 μM Z-FY-CHO CatL inhibitor, 5 μg/ml MSKE, and 20 μg/ml MSKE significantly decreased the amounts of active CatL.

The novelty of this invention is that it is using a natural plant product, Muscadine Grape Skin Extract that has never before been tested in preventing the formation of mature osteoclasts which are important in bone resorption and bone metastasis. Therefore, this compound or its structural analogs may be employed in destroying prostate cancer cells which may prevent bone metastasis. Since its cytotoxic effects are effective in the more aggressive prostate cancer cell lines overexpressing Snail, which may represent the form of cancer in those with bone metastasis, this compound may be of therapeutic value in aggressive prostate and breast cancer which are highly metastatic.

Breast and prostate cancer are a leading cause of cancer death among women and men, with the skeleton the preferred site for metastasis. Osteoblastic lesions, characteristic of prostate cancer, are caused by an excess of osteoblast activity relative to resorption by osteoclasts, leading to abnormal bone formation. In breast cancer, osteolytic lesions are found in 80% of patients with stage IV metastatic disease. The lesions are characterized by increased osteoclast activity and net bone destruction. The primary cause of prostate and breast cancer death is metastasis. The current treatment options for prostate and breast cancer have various side effects that are resulting in greater morbidity and mortality. Numerous studies have shown an association between reduced cancer risk and intake of a diet rich in fruits and vegetables. Hence, a more complete understanding of the molecular mechanisms through which MSKE or related structures act on cellular processes involved in prostate cancer progression could lead to prevention and treatment of prostate cancer. This compound or its related structures could be employed clinically either individually or in combination with currently used chemotherapeutic agents, in the treatment of bone metastasis due to prostate or breast cancer progression.

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric.

### Materials and Methods

### MSKE

MSKE was obtained through the process taught in Hudson et al., “Inhibition of prostate cancer growth by muscadine grape skin extract and resveratrol through distinct mechanisms” Cancer Res. 2007, 67(17): 8396-8405. This reference is specifically incorporated in its entirety herein. MSKE is obtained from the muscadine grape Vitis rotundifolia and the predominant ingredients are anthocyanin 3,5-diglucosides, ellagic acid, and ellagic acid precursors. MSKE contains no significant amount of resveratrol.

### Cell Culture, Reagents, and Antibodies

C4-2 and ARCaP-epithelial (ARCAP-E) human prostate cancer cells were a kind gift from Dr Leland Chung (Cedars Sinai Medical Center, Los Angeles, Calif.). LNCaP and MCF-7 cells were obtained from ATCC. ARCaP-E cells were stably transfected with constitutively active Snail cDNA as has been described previously. The MCF-7 cells stably transfected with empty Neo vector (MCF-7 Neo) or constitutively active Snail (MCF-7 Snail) were kindly provided by Dr. Mien-Chie Hung, The University of Texas MD Anderson Cancer Center, Houston Tex., and established as described previously. C4-2 cells transduced with Snail shRNA for stable Snail knockdown has been described previously. Cells were grown in RPMI supplemented with 10% fetal bovine serum and 1× penicillin-streptomycin (LNCaP, C4-2 MCF-7 transfectants) or in T-media supplemented with 10% fetal bovine serum and 1× penicillin-streptomycin (ARCaP-E transfectants) and kept at 37° C. with 5% CO₂ in a humidified incubator. Anti-mouse α-tubulin antibody and TRAP staining kit was from Sigma-Aldrich, Inc., St Louis, Mo. Rat monoclonal anti-Snail antibody, anti-p-STAT-3, HRP-conjugated goat anti-rat antibodies were from Cell Signaling Technology, Inc., Danvers, Mass. CatL antibody, Recombinant mouse Macrophage-Colony Stimulating Factor (M-CSF), CatL specific inhibitor (Z-FY-CHO), and Osteoprotegerin (OPG) were purchased from R&D Systems (Minneapolis, Minn.). The donkey-Ig goat and STAT-3 antibodies were purchased from Santa Cruz. HRP-conjugated sheep anti-mouse and sheep anti-rabbit were purchased from Amersham Biosciences, Buckinghamshire, UK. Luminata Forte HRP chemiluminescence detection reagent was purchased from EMD Millipore (Billerica, Mass.). The protease inhibitor cocktail was from Roche Molecular Biochemicals, Indianapolis, Ind. from BD Biosciences, San Jose, Calif.

### Western Blot Analysis

Cells were lysed in a modified RIPA buffer (50 mM Tris pH 8.0, 150 mM NaCl, 0.02% NaN₃, 0.1% SDS, 1% NP-40, 0.5% sodium deoxycholate) containing 1.5× protease inhibitor cocktail, 1 mM phenylmethylsufonyl fluoride (PMSF), and 1 mM sodium orthovanadate. Whole cell lysates were freeze-thawed at −80° C./4° C. for three cycles, then centrifuged at 13,000 rpm for 30 min at 4° C. Supernatants were collected and quantified using a micro BCA assay (Promega, Madison, Wis.). 40 μg of cell lysate was resolved using 10% SDS PAGE, followed by transblotting onto nitrocellulose membrane (Bio-Rad Laboratories, Hercules, Calif.). Membranes were blocked with 3% milk (TBS-T containing 3% milk), then washed and incubated with primary antibody dilution buffer. After washing, the membranes were incubated in peroxidase-conjugated sheep anti-mouse, or anti-goat, anti-rat IgG, washed, and visualized using Luminata Forte ECL reagent (Millipore, Billerica, Mass.). The membranes were stripped using Restore Western blot stripping buffer (Pierce Biotechnology, Inc., Rockford, Ill.) prior to re-probing with a different antibody. For treatments, 70% confluent cells were serum-starved in phenol red-free serum-free RPMI containing penicillin/streptomycin for 24 h prior to treatment with MSKE or Z-FY-CHO in phenol-free serum-free RPMI containing 5% FBS DCC-FBS for 3 days.

### Zymography

We utilized the cathepsin zymography technique as previously described. Briefly, 1 mL of conditioned media (CM) containing 0.1 mM leupeptin was concentrated utilizing vivaspin tube (GE Health Care). The concentrated CM was diluted by adding Sul of the sample to 45 μl of 1× RIPA buffer followed by determination of the protein concentration using BCA protein assay kit. Gelatin (0.2%) was utilized as the zymography substrate and CM was electrophoresed followed by incubation in cathepsin renaturing buffer, incubation in pH 6 sodium phosphate assay buffer and overnight incubation at 37° C. with the assay buffer. The gel was stained with Coomassie blue for one hour and then destained. The enzymes hydrolyze the embedded substrate in situ, and proteolytic activity can be visualized as cleared bands. Cathepsin activity was subsequently quantified using densitometry (NIH Image J).

Transfection with STAT-3 Short Interfering RNA (siRNA)

ARCaP Snail and C4-2 (5×10⁵ cells per well) were plated in 6-well plates in complete growth media and left overnight for attachment. The next day, STAT3 siRNA (Dharmacon, Inc.)

transfections were performed according to manufacturer instructions. The STAT3 siRNA are pooled from four On-Target plus SMARTpool siRNA with the following identities and target sequences; J-003544-07, target sequence: GAGAUUGACCAGCAGUAUA, J-003544-08, target sequence: CCAACAAUCCCAAGAAUGU, J-003544-09, target sequence: CGAAAGGUCAGAUCAACAA, J-003544-10, target sequence: CAACAGAUUGCCUGCAUUG. Briefly, the cells were washed with sterile Phosphate Buffered Saline (1× PBS) followed by addition of 200 nM control or STAT-3 siRNA in serum-free RPMI. The cells were then incubated at 37° C., 5% CO₂ for 5 hours after which the media was replaced with 2 ml of 5% DCC followed by incubation at 37° C., 5% CO₂ for 72 hours. Cell lysates were then harvested and western blot analysis performed to probe for STAT-3 and p-STAT while CM was collected for zymography to determine the CatL activity.

### Ethical Statement Related to the Use of Human Breast Tumor Samples

Prostate tumors, and matched normal tissues were obtained from the following sources-a) Protein biotechnologies, Ramona, Calif.; b) US Biomax, Inc. (Catalog #?, Rockville, Md.). Protein Biotechnologies Inc. provides pharmaceutical, biotechnology, government, and academic institutions with human clinical specimen derivatives. Tissues are obtained through a global network of participating medical centers that employ IRB approved protocols and strict ethical guidelines to ensure patient confidentiality and safety. Identical procedures are used to prepare all patient samples. Specimens are flash frozen to −120° C. within 5 min of removal to minimize autolysis, oxidation, and protein degradation. Tissue specimens are homogenized in modified RIPA buffer (PBS, pH 7.4, 1 mM EDTA, and protease inhibitors) to obtain the soluble proteins, and centrifuged to clarify.

### Immunohistochemistry

Examination of the expression and distribution of CatL in human prostate cancer was performed by immunohistochemistry (IHC) using tissue microarray. IHC was performed using the Avidin-biotin immunohistochemical method. The microarray was deparaffinised in xylene and rehydrated using alcohol. Endogenous peroxidase activity was blocked by 3% hydrogen peroxide. After antigen retrieval, sections were incubated with 10% serum to avoid the non-specific binding. Sections were incubated with 1:200 primary antibody against CatL at 4° C. overnight followed by biotinylated secondary antibody, and incubation with avidin-biotin complex (Vector). Immunoreactivity was visualized using diaminobenzidine (Sigma-Aldrich, St. Louis, Mo., USA). The slide was subsequently counterstained with hematoxylin and mounted with xylene solution. Images were acquired using the Axiovision Rel 4.8.

### In Vitro Cell Migration Assay

We utilized Costar 24-well plates containing a polycarbonate filter insert with an 8-μm pore size, to coat with 4.46 μg/μl rat tail collagen I on the outside for 24 h at 4° C. 5×10⁴ cells were plated in the upper chamber containing RPMI supplemented with 0.1% fetal bovine serum (FBS) while the lower chamber contained RPMI supplemented with 10% FBS. After 5 h, cells that migrated to the bottom of the insert were fixed, stained with 0.05% crystal violet, and counted to obtain the relative cell migration.

### In Vitro Cell Invasion Assay

The invasive properties of the cell lines were measured using the BD BioCoat Matrigel Invasion guidelines. Briefly, Boyden chamber inserts (Thermo Fisher Scientific, Waltham, Mass., USA) were coated with 40 μl 1:4 Matrigel and allowed to solidify at 37° C. for 1 h. 5×10⁴ cells were seeded in triplicate in 0.1% FBS, while the lower chamber contained 10% FBS. Cells were allowed to invade through the porous membrane coated with Matrigel at 37° C. for 24-72 h. Inserts were fixed, stained with 0.05% crystal violet. Cell counts were performed for the determination of relative cell invasion.

### In Vitro Osteoclastogenesis Assay

3×10³ ARCaP-Neo/MCF-7 Neo or ARCaP-Snail/MCF-7 Snail was co-cultured with 40×10⁴ spleen macrophages in 48-well plates plus 1 ng/ml M-CSF plus or minus 5 μg/mL MSKE, 20 μg/mL MSKE or 504 Z-FY-CHO Cat L inhibitor. The cells were fed every three days by replacing half the media with fresh media plus or minus MSKE or Z-FY-CHO. Macrophages alone were utilized as a negative control. After 7-14 days the cells were fixed with 3% formaldehyde and processed for TRAP staining according to manufacturer instructions, to visualize the formation of mature osteoclasts.

### Statistical Analysis

Data were presented as the mean±SD from three independent experiments. Data analysis for statistical significance was done using Student's t-test. P-value was less than 0.05, indicating statistical significance of the data.

### Results

### CatL is Increased in Patient Prostate and Breast Tumor Tissue

CatL has been shown to be increased in patient prostate and breast tissue. To confirm these findings we stained for CatL by IHC using prostate tumor tissue microarray and analyzed CatL expression by western blot using patient breast tissue. Normal prostate epithelial tissues expressed low levels of Cat L in the cytoplasm. Alternatively, higher levels of Cat L were detected within prostate adenocarcinoma which increased with tumor grade. Moreover, CatL expression was predominantly cytoplasmic in stage II and III whereas it was both nuclear and cytoplasmic in stage IV and exclusively nuclear in bone metastatic tissue. However, CatL staining was low in cancer cells that metastasized to the abdominal wall.

Using normal/tumor-matched breast cancer, we analyzed the expression of Cat L by western blot analysis. The tumor lysates expressed higher levels of mature Cat L as compared to normal tissue. This shows that Cat L expression increases with tumor progression in breast and prostate cancer.

Snail is Associated with Increased CatL Activity in Prostate and Breast Cancer Cell Lines

Since Snail and CatL are both involved in bone turnover, we speculated that Snail may regulate CatL expression/activity. We therefore examined the expression and activity of CatL by western blot analysis and zymography, respectively, in either C4-2 prostate cancer cells with stable Snail knockdown that exhibits decreased cell migration as previously reported or MCF-7 breast, LNCaP prostate and ARCaP-E prostate Snail-overexpressing cells that represent an EMT model as previously reported.

Lysates for Western blot analysis were prepared as described in the Material and Methods section. 40 ug of protein were electrophoresed using 10% SDS-page, followed by western blotting on nitrocellulose for Snail and CatL. Alpha tubulin was used as a loading control. CatL expression is represented by three bands (pre-pro Cathepsin L, pro-cathepsin L, and mature Cathepsin L).

Preparation of lysates for zymogram are described in the materials and methods section. 16 μg of condition media were loaded for cathepsin gelatin zymography and incubated overnight in phosphate buffer pH 6 prior to staining with Coomassie blue and destained to visualize bands (white).

Cathepsin L activity was quantified with densitometry of each band on the gel. (n=3, ***p<0.001, **p<0.01, *p<0.05). Values were normalized to untreated controls and the mean±SEM of data were obtained from three independent replicate experiments. Statistical analysis was done with Student t test

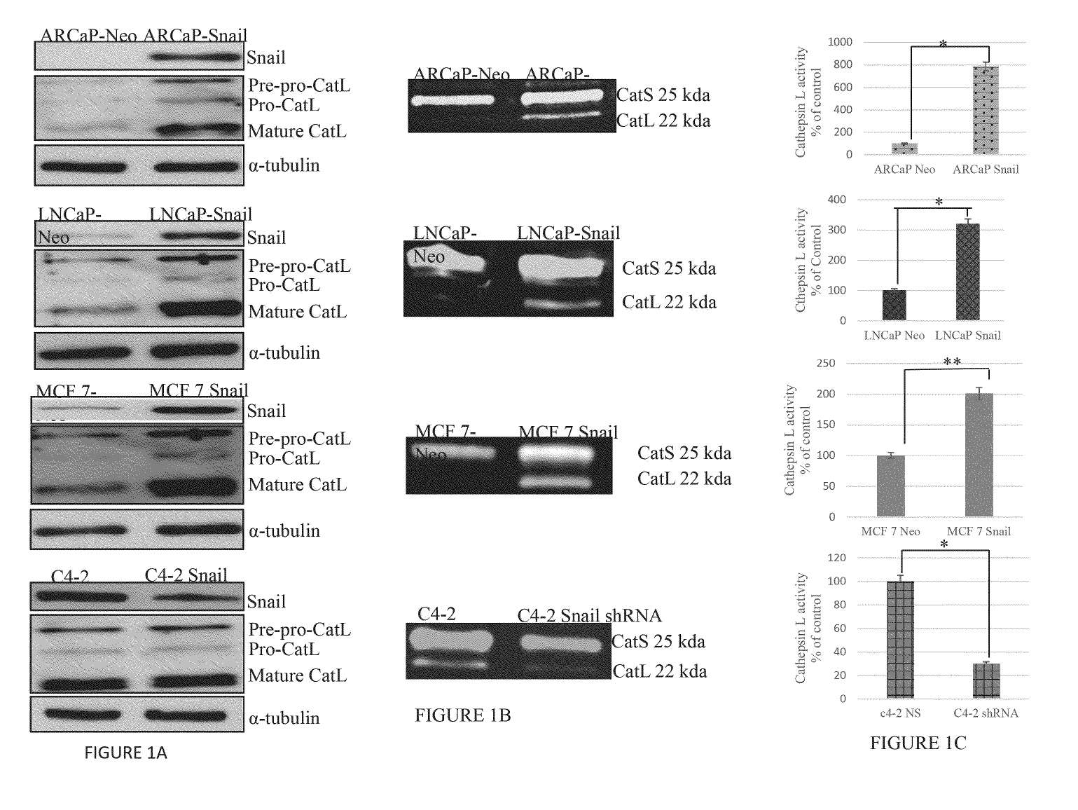

As seen in FIG. 1A, western blot analysis showed that immature (pre-pro and pro) as well as mature CatL expression were higher in the MCF-7, ARCaP and LNCaP Snail-transfected cells compared to the Neo controls but not significantly altered in C4-2 control (C4-2 NS) or C4-2 cells with stable Snail knockdown (C4-2 E8). However, C4-2 cells that have been stably transduced with shRNA to knockdown Snail expression (C4-2 E8) displayed a decrease in Cat L activity compared to the empty vector C4-2 NS as shown by zymography (FIGS. 1B and 1C), while MCF-7, LNCaP, and ARCaP cells stably transfected with Snail cDNA displayed increased amount of active CatL. This demonstrates that Snail can positively regulate Cat L expression and activity.

### STAT3 Regulates CatL Activity in Prostate Cancer Cells

Since we observed that cells transfected with Snail are associated with an increase in CatL activity, we wanted to examine the signaling pathway by which Snail may regulate CatL activity. Since STAT-3 signaling pathway has been shown to regulate Snail via Liv-1 and also regulate CatL activity, we tested the hypothesis that the STAT-3 pathway was involved, by utilizing siRNA against STAT-3 to determine the effects of STAT-3 knockdown on CatL activity.

C4-2 and ARCaP-E cells overexpressing Snail were transiently transfected with control siRNA or STAT-3 siRNA and the expression levels of Snail and mature CatL were determined with western blot. Cathepsin zymography was performed to determine mature CatL activity. In both cells there was a decrease in the expression of Snail, as well as a decrease in mature CatL expression and activity.

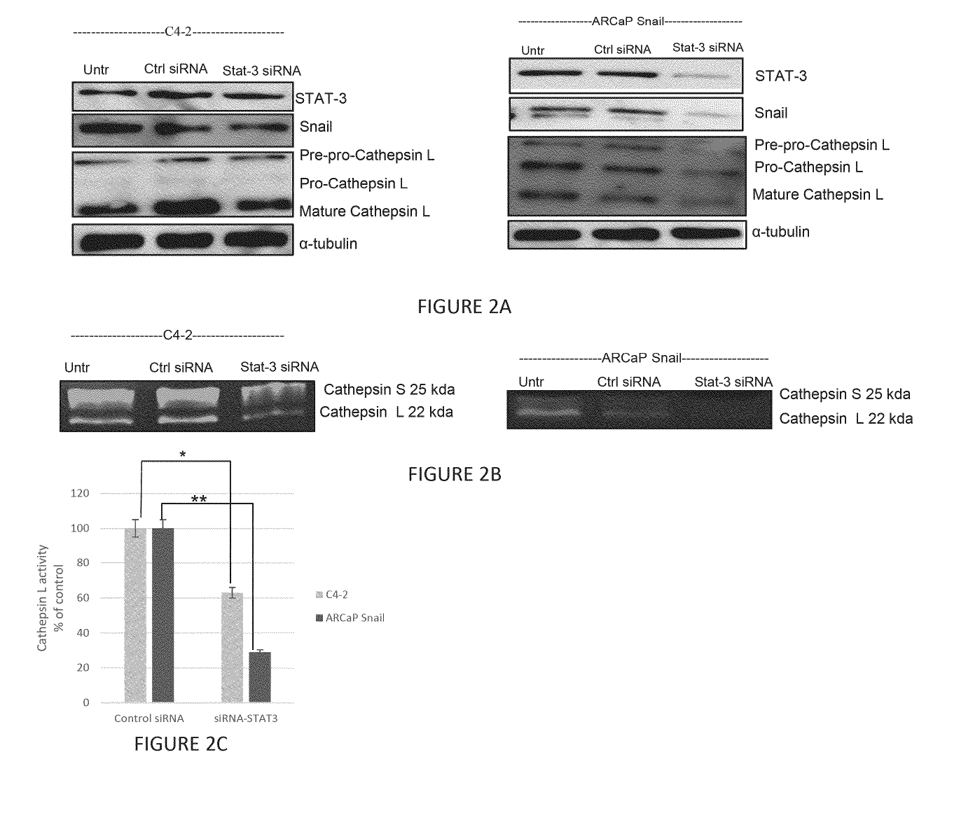

Cathepsin L activity was quantified with densitometry of each band on the gel. Asterisks represent p values of statistical significance. (n=3, ***p<0.001, **p<0.01, *p<0.05). Values were normalized to untreated controls and the mean±SEM of data were obtained from three independent replicate experiments. Statistical analysis was done with Student t test. We found that STAT-3 knockdown in ARCaP prostate cancer cells overexpressing Snail and C4-2 prostate cancer cells led to decreased Snail and mature CatL expression (FIG. 2A) as well as decreased amounts of active CatL (FIGS. 2B, 2C). This shows that the JAK/STAT pathway may be involved in Snail activation of CatL.

MSKE and Z-FY-CHO Decreased Snail Expression and CatL Activity in MCF-7 and ARCaP Cells Transfected with Snail, in Part Via Inhibition of Active STAT-3

Next, we examined whether Snail/CatL signaling could be antagonized by natural or pharmacological products. MSKE has recently been shown to promote apoptosis of prostate cancer cells, but not normal cells at 20 μg/ml. It has al can revert EMT by causing a decrease in the expression of vimentin and re-expression of E-cadherin. Z-FY-CHO, a potent and reversible selective inhibitor of Cat L, has been shown to inhibit bone resorption in rat bone cells by inhibiting collagen degradation (Woo JT, Eur J Pharmacol, 1996). C4-2 prostate cancer cells or ARCaP-E (prostate) and MCF-7 (breast) cancer cells overexpressing Snail were treated with either Z-FY-CHO or MSKE for up to 72 h.

Cells were treated with 5 μg/mL or 20 μg/mL MSKE and 5 μM Z-FY-CHO for 72 hours. Lysates for Western blot analysis were prepared as described in Material and Methods section.

The expression levels of Snail, mature CatL, STAT-3 and Phospho-STAT-3 (pstat-3) were determined with western blot. Alpha tubulin was used as a loading control. Across cell lines there is a decrease in Snail, CatL and STAT-3 phosphorylation (pstat-3), compared to controls. Mature Cathepsin L activity was determined by gelatin zymography. Cathepsin L activity was quantified with densitometry of each band on the gel. The quantification of CatL activity shows that the treatments with MSKE and Z-FY-CHO significantly decreased the amount of active CatL compared to the untreated controls. Asterisks represent p-value of statistical significance (n=3, ***p<0.001, **p<0.01, *p<0.05). Values were normalized to untreated controls and the mean±SEM of data were obtained from three independent replicate experiments. Statistical analysis was done with Student t test.

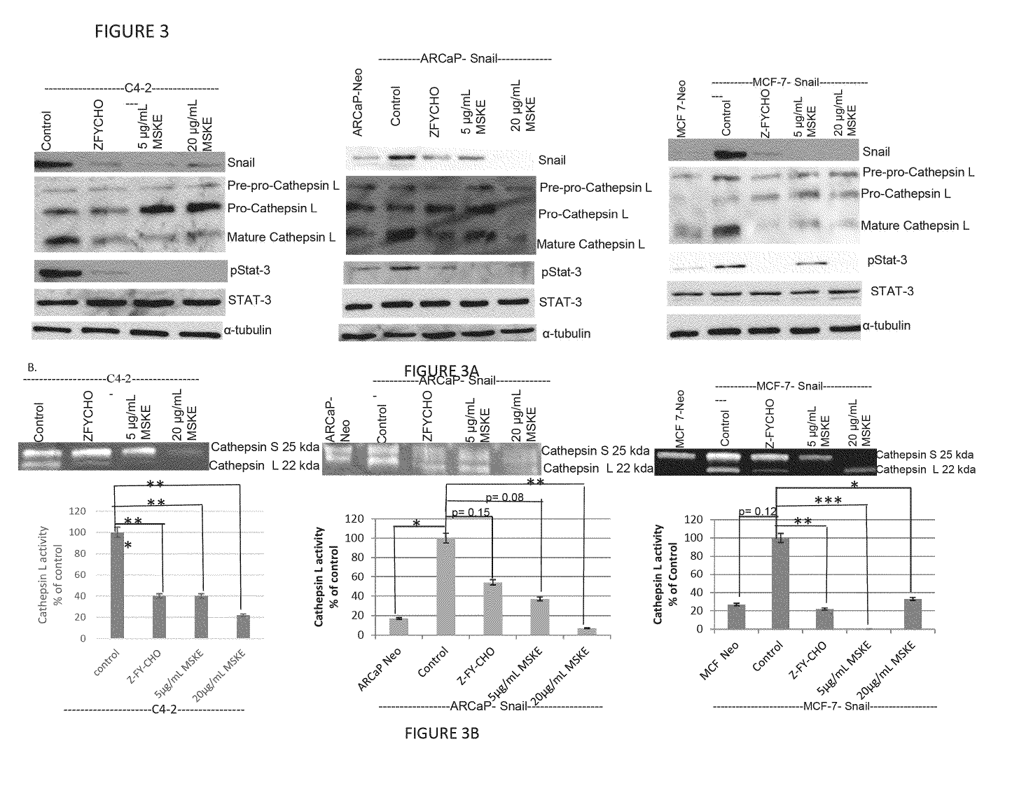

We observed that 5 and 20 μg/mL MSKE led to a decrease in STAT-3 activation (p-STAT-3), Snail and mature CatL expression and CatL activity in all cell lines tested, with 20 μg/mL showing the highest effect (FIGS. 3A and 3B). This suggests that MSKE may antagonize CatL activity via inhibition of STAT-3 and Snail signaling. Interestingly, Z-FY-CHO (CatL inhibitor) could decrease the level of STAT-3 activation, Snail expression, and Cat L expression/activity. This suggests that although Snail can regulate CatL activity via STAT-3, a positive feedback loop exists, whereby CatL can regulate Snail possibly via STAT-3 signaling. This signaling can be effectively inhibited by MSKE and Z-FY-CHO.

### MSKE and Z-FY-CHO Antagonize Snail-Mediated Cell Migration and Invasion

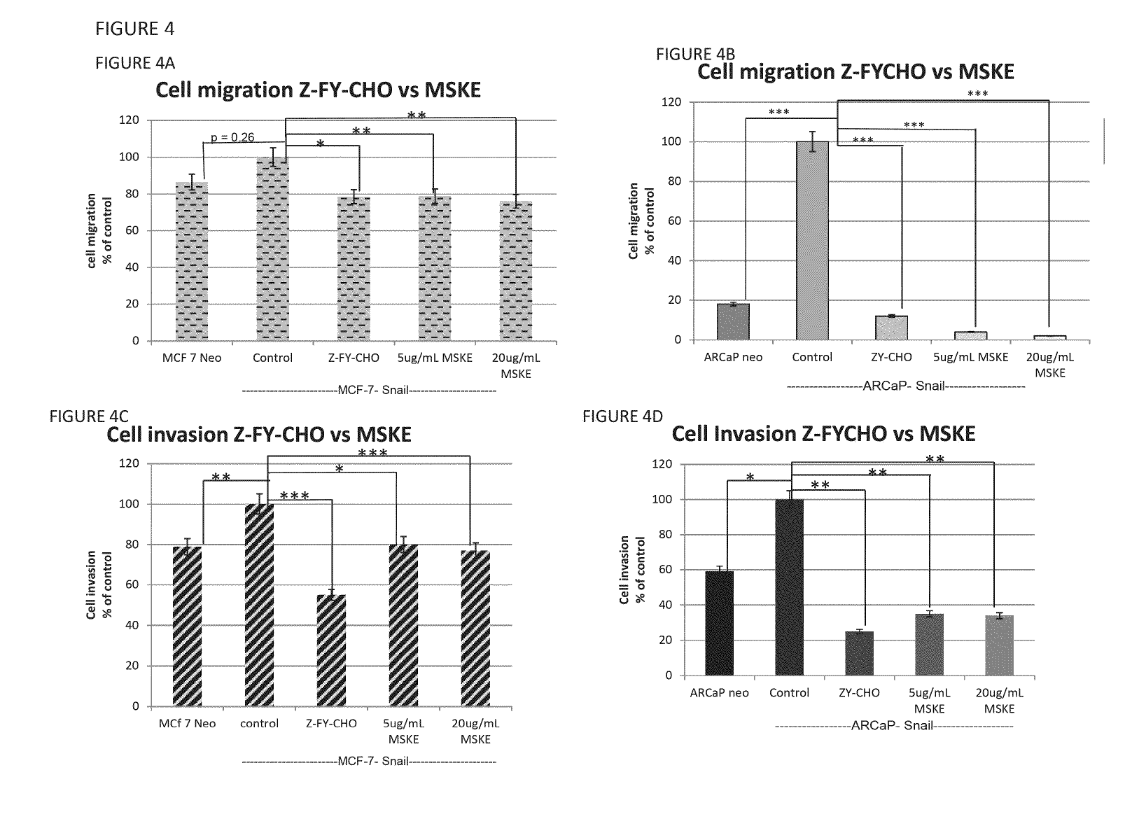

Next we examined if CatL mediates functional effects of Snail and whether Snail-mediated cell migration and invasion can be antagonized by CatL inhibitor or MSKE. ARCaP-E prostate and MCF-7 breast cancer cells overexpressing Snail showed increase in migration and invasion, as compared to empty vector Neo controls, which could be abrogated upon treatment with MSKE or Z-FY-CHO (FIG. 4A-D).

FIGS. 4A and 4B show MCF-7 and ARCaP-E cells over expressing Snail treated with 5 μg/mL, 20 μg/mL Muscadine Grape Skin Extract (MSKE) and 5 μM Z-FY-CHO for 72 hours. 5×10⁴ cells were plated on transwell coated with Type-I collagen as described in the materials and methods section. The cells were allowed to migrate for 5 hours and then fixed and stained. The number of cells that migrated through the transwell membranes was determined by counting at least 4 random microscopic fields.

FIGS. 4C and 4D represent cell invasion of the same cell lines. 5×10⁴ cells were plated on transwells coated with Matrigel. Cell invasion was allowed to take place for 24 hours. The number of cells that invaded through the transwell membranes were determined by counting at least 4 random microscopic fields. Compared to neo empty vector cells overexpressing Snail represent an increase in migration and invasion. MSKE and Z-FY-CHO decrease migratory and invasive cells. Asterisks represent p-value of statistical significance (n=3, ***p<0.001, **p<0.01, *p<0.05). Values were normalized to untreated controls and the mean±SEM of data were obtained from three independent replicate experiments with at least three wells per treatment group in each individual replicate. Statistical analysis was done with Student t test.

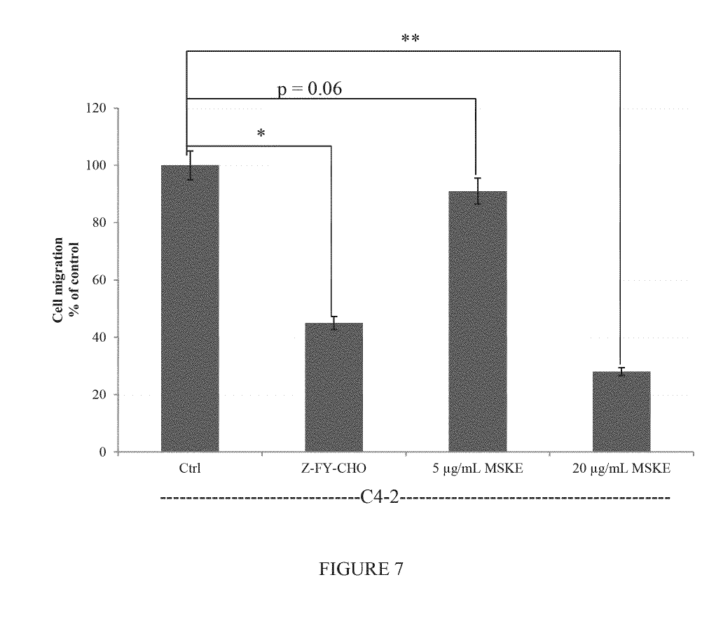

Similarly, cell migration in C4-2 parental prostate cancer cells displayed decreased cell migration upon treatment with MSKE (FIG. 7). Therefore, MSKE and Z-FY-CHO can antagonize Snail-mediated cell migration and invasion.

### MSKE and Z-FY-CHO Antagonize Snail-Mediated Osteoclastogenesis

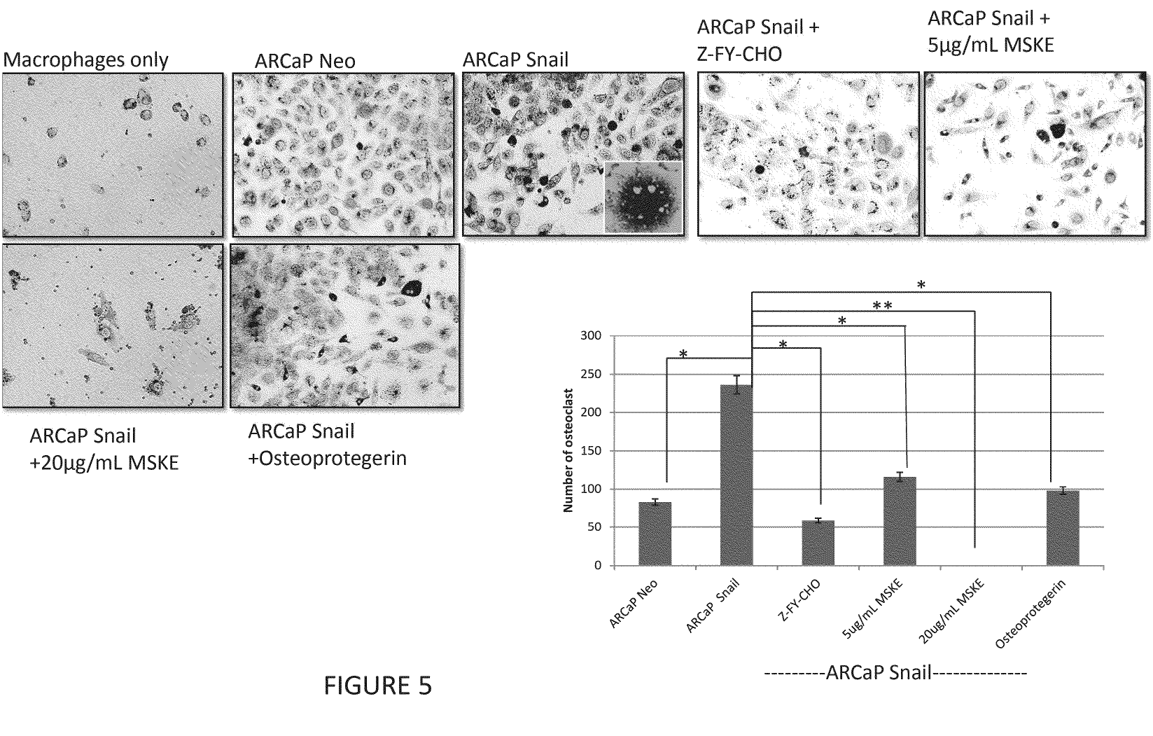

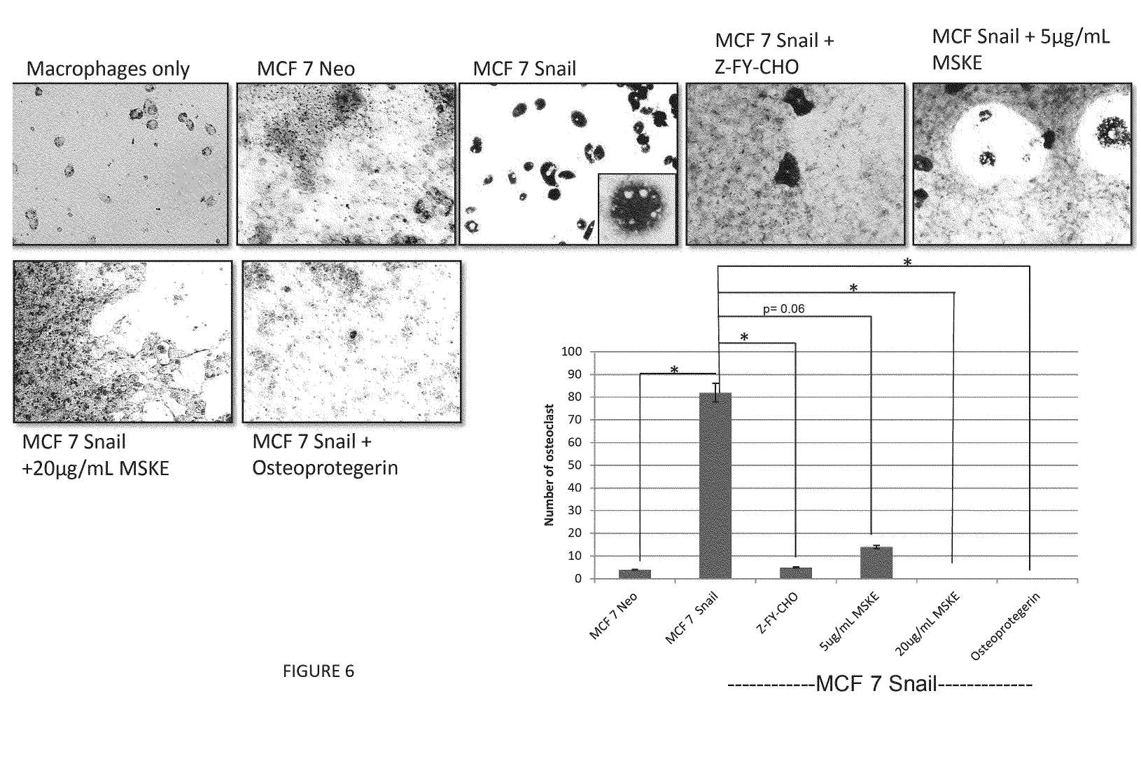

Since we have previously shown that Snail can increase osteoclastogenesis and CatL has been shown to be involved in bone resorption, we investigated whether CatL may be involved in Snail-mediated osteoclastogenesis, and whether this biological function can be antagonized by MSKE. Our results indicated that compared to the control Neo empty vector-expressing cells, the Snail transfected ARCaP and MCF-7 cells displayed significant increase in the formation of mature osteoclasts as seen by TRAP staining (FIG. 5 and 6).

3×10³ or 1×10³ prostate cancer cells overexpressing Snail (see FIG. 5) and breast cancer cells overexpressing Snail (FIG. 6) were co-cultured with 40×10⁴ Macrophages for 14 days and treated with 50 ng/mL osteoprotogerin (control), 5 μM Z-FY-CHO, 5 μg/mL or 20 μg/ML MSKE. Macrophages only were also used as a control. 250 uL of media with 1 ng/mL Macrophage Colony factor plus or minus treatments were changed every three days. TRAP staining showed the formation of osteoclast (purple) and the cells are yellow. Osteoclasts were determined by having 3 or more nuclei (FIGS. 5 and 6 insets). Graphical representation of the number of osteoclasts are shown as mean±SEM of data were obtained from three independent replicate experiments with at least three wells per treatment group in each individual replicate. The treatments 5 μM Z-FY-CHO, 5 μg/mL or 20 μg/ML MSKE were shown to significantly decrease the formation of osteoclasts compared to the untreated controls. Neo empty vectors were also used as a control. Asterisks represent p-value of statistical significance (n=3, ***p<0.001, **p<0.01, * p<0.05).Statistical analysis was done with Student t test.

Moreover, 5 μM Z-FY-CHO and 5 μg/mL and 20 μg/ml MSKE significantly abrogated Snail-mediated osteoclastogenesis. This suggests that Snail mediates osteoclastogenesis in part via CatL activity, which can be inhibited by MSKE and Z-FY-CHO.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A method of decreasing Snail expression in cells comprising contacting the cells with Muscadine Grape Skin Extract (MSKE).

2. The method of claim 1, wherein the method further decreases CatL activity and STAT-3 activity in the cells.

3. The method of claim 1, wherein the method decreases CatL expression.

4. The method of claim 1, wherein the cells are cancer cells overexpressing Snail and the method inhibits the increased migration and invasion and osteoclastogenesis caused by cancer cells overexpressing Snail.

5. The method of claim 1, wherein the method inhibits the formation of mature osteoclasts.

6. The method of claim 1, wherein the method further results in decreased STAT-3 and p-STAT-3 expression.

7. The method of claim 1, wherein the MSKE contains anthocyanin 3,5-diglucosides, ellagic acid, and ellagic acid precursors.

8. The method of claim 7, wherein the MSKE does not contain significant amounts of resveratrol.

9. A method of treating bone metastatic disease in a subject comprising administering MSKE to the subject.

10. The method of claim 9, wherein the MSKE contains anthocyanin 3,5-diglucosides, ellagic acid, and ellagic acid precursors.

11. The method of claim 10, wherein the MSKE does not contain significant amounts of resveratrol.

12. The method of claim 9, wherein the MSKE causes a reduction in Snail expression of cells of the subject.

13. The method of claim 9, wherein the MSKE decreases CatL activity and STAT-3 activity in the cells.

14. The method of claim 9, wherein the MSKE decreases CatL expression.

15. The method of claim 9, wherein the MSKE inhibits increased migration and invasion and osteoclastogenesis of cancer cells that cause bone metastatic disease.

16. The method of claim 9, wherein the MSKE inhibits the formation of mature osteoclasts.
